(19) United States
(12) Patent Application Publication
(10) Pub. No.: US 2013/0209442 A1
(43) Pub. Date: Aug. 15, 2013

(54) WOUND CLEANING CHEMICAL COMPOSITION AND METHOD FOR MANUFACTURING

(71) Applicant: Luis R. NOVOA, San Juan, PR (US)

(72) Inventor: Luis R. NOVOA, San Juan, PR (US)

(21) Appl. No.: 13/758,676

(22) Filed: Feb. 4, 2013

Related U.S. Application Data
(60) Provisional application No. 61/594,057, filed on Feb. 2, 2012.

(51) Int. Cl. A61K 38/48; A61K 31/045; A61K 33/14; A61K 33/20

(52) U.S. Cl. CPC A61K 38/482; A61K 33/20; A61K 31/045; A61K 33/14
USPC 424/94.64

(57) ABSTRACT

A wound cleaning chemical composition that includes an unpurified saline solution in the range of 1.5 to 3 grams per cc, a lactoferrin solution in the range of 0.001% to 1.5% by volume, an ethanol solution in the range of 0.05% to 1.5% by volume, a sodium bicarbonate solution in the range of 1.2% to 5.1% by volume and a hypochlorite solution in the range of 0.06% to 4.7% by volume. The wound cleaning chemical composition also includes a method for manufacturing a wound cleaning chemical composition that includes the steps of preparing an unpurified saline solution, adding a hypochlorite solution, an ethanol solution and a sodium bicarbonate solution to the unpurified saline solution and adding a lactoferrin solution.

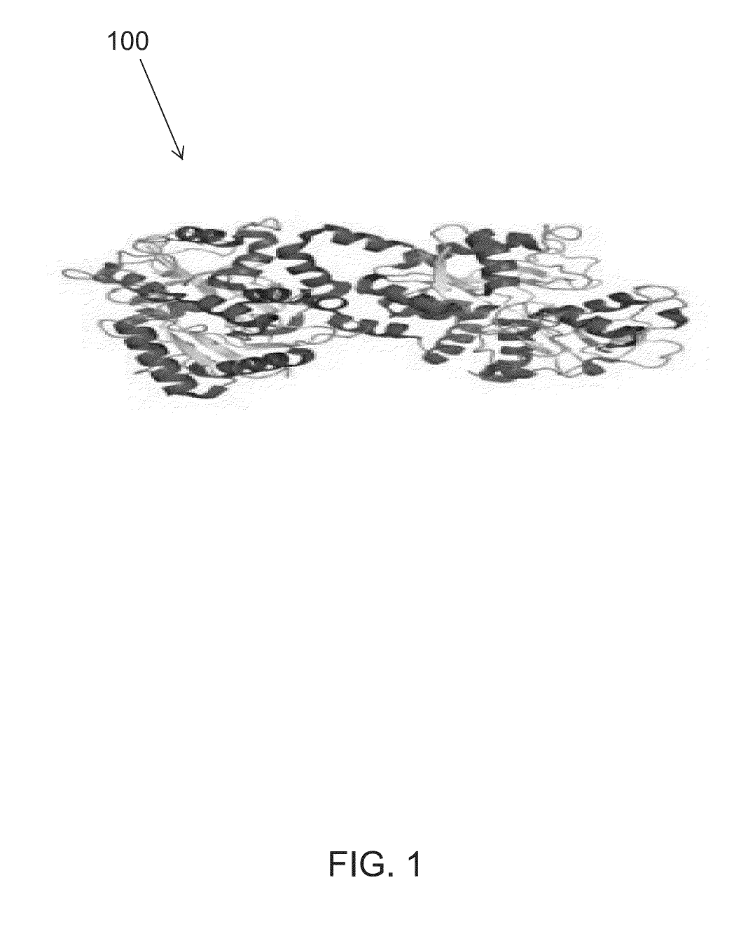

This application claims priority to U.S. Provisional Application 61/594,057 filed on Feb. 2, 2012, the entire disclosure of which is incorporated by reference.

### BACKGROUND OF THE INVENTION

1. Field of the Invention

The present invention is a chemical composition. More specifically, the present invention is a wound cleaning chemical composition.

2. Description of the Related Art

There have been many solutions for the treatment and cleansing of abrasions, lacerations, ulcers and other types of wounds. Most of these in the medical community are single agents, or mixtures of chemicals or variants of saline solution with the addition of one or single enzymes or one or chemicals to achieve a desired treatment and cleansing. Most of these solutions are pharmaceuticals that require health insurance, one or more prescriptions or are just very expensive and not natural.

### BRIEF SUMMARY OF THE INVENTION

The present invention is a chemical composition. More specifically, the present invention is a wound cleaning chemical composition.

The present invention also relates to the field of wound cleansers, topical cleansers or topical skin cleansers. More specifically it relates to a solution which mimics the properties of lacrimal tears which contains substances with antibacterial and cleansing properties, that when applied to the wound or affected area of the skin helps in the cleansing and healing process. The mixture includes purified water, lactoferrin, salt, minerals, calcium hypochlorite, bicarbonate and ethanol.

The method of manufacturing a wound cleaning chemical composition includes the production of a pH balanced solution of hypochlorite or CaCl solution, ethanol, bicarbonate and salt. These components are mixed, heated and then tested for pH, osmolality and bacterial count. After the mixture is done liquid lactoferrin is added.

The method of manufacturing a wound cleaning chemical composition more specifically includes preparing a saline solution in the range of 1.5 to 3.0 g/cc, a lactoferrin solution in the range of 0.001% to 1.5% by volume, an ethanol solution in the range of 0.05% to 1.5% by volume, a sodium bicarbonate solution in the range of 1.2% to 5.1% by volume and a hypochlorite solution in the range of 0.061% to 4.7% by volume.

It is an object of the present invention to provide a wound cleaning chemical composition that has a balanced formula with a naturally occurring enzyme produced by human tears combined with saline solution with trace elements and minerals in controlling bacterial growth and help in the healing and cleansing of the skin of both humans and animals.

It is an object of the present invention to provide a wound cleaning chemical composition that is an affordable over-the-counter formulation with natural well known ingredients to help cleanse and heal wounds.

It is an object of the present invention to provide a method for manufacturing a wound cleaning chemical composition.

### DETAILED DESCRIPTION OF ILLUSTRATIVE EMBODIMENTS

Various aspects of the illustrative embodiments will be described utilizing terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

Lactoferrin is a commercially available product. It's a globular glycoprotein represented in various secretory fluids such as milk, saliva, tears and nasal secretions. Lactoferrin is well-known for its antibacterial, antiviral, antiparasitic, catalytic, anticancer and antiallergenic properties.

Sodium chloride in an unpurified form contains natural minerals and elements that mimic those of tears, secretions and blood serum, in higher concentrations such as above 1.5 g/cc of water. Sodium chloride is a well-known preservative, lowering bacterial, viral and parasitic colonization, so an unpurified sodium chloride solution is also a good wound cleanser. Calcium hypochlorite is a well-known cleanser, antibacterial, antiviral and naturally occurring preservative and if utilized in balanced diluted concentrations is harmless to human and animal skin and wounds. Saline hypochlorite solutions have been utilized for decades in the medical field for the treatment of diabetic ulcers and have been well-documented in medical and surgical literature. Ethanol is also commercially available and is also a well-known preservative and antiseptic and a well-suited choice for a wound cleanser. Bicarbonate is also found commercially and is utilized for its pH balancing properties to avoid burning any affected areas.

A combination of a natural enzyme salt such as lactoferrin with minerals and trace elements mimic the lacrimal tears in fighting bacterial, viral and parasitic colonization found in wounds, lacerations, abrasions or disrupted skin. In combination with the calcium hypochlorite and the ethanol, the lactoferrin produces a solution to help cleanse human skin or animal skin with a balanced solution, in an affordable and over the counter formulation.

Table 1 provides a specific breakdown of the chemical components of the wound cleaning chemical composition.

TABLE 1

| | |
|---|---|
| Unpurified Saline Solution | In the range of 1.5 to 3 Grams Per CC |
| Lactoferrin Solution | In the range of 0.001% to 1.5% by Volume |
| Ethanol Solution | In the range of 0.05% to 1.5% by Volume |
| Sodium Bicarbonate Solu- | In the range of 1.2% to 5.1% by Volume |
| tion | |
| Hypochlorite Solution | In the range of 0.06% to 4.7% by Volume |

The method for manufacturing a wound cleaning chemical composition includes a pH balanced solution of hypochlorite (Calcium chloride or CaCl solution), ethanol, bicarbonate and sodium chloride. These components are mixed, heated and then tested for pH, osmolality and bacterial count. After the pH balanced solution of hypochlorite (CaCl solution), ethanol, bicarbonate and salt is processed, lactoferrin is added.

The method includes the addition of an unpurified saline solution in the range of 1.5 to 3.0 gs/cc, a hypochlorite solution in the range of 0.06% to 4.7% by volume, an ethanol solution in the range of 0.05% to 1.5% by volume and a sodium bicarbonate solution in the range of 1.2% to 5.1% by volume. The unpurified saline solution in the range of 1.5 to 3.0 gs/cc, a hypochlorite solution in the range of 0.06% to 4.7% by volume, an ethanol solution in the range of 0.05% to 1.5% by volume and an sodium bicarbonate solution in the range of 1.2% to 5.1% by volume forms a solution that is mixed, pasteurized and then tested for pH, osmolality and bacterial count. The sodium bicarbonate solution in the range of 1.2% to 5.1% by volume is utilized to adjust the pH of the wound cleaning chemical composition. The wound cleaning chemical composition is tested to achieve a pH in the range of 7.3 pH to 7.45 pH and is then cooled. The lactoferrin solution is in the range of 0.001% to 1.5% by volume. The formed solution is then let rest. The wound cleaning chemical composition is then tested in bacterial petri dishes or any other suitable method for potential bacteria growth or contamination. The wound cleaning chemical composition can also be bottled and sealed or packaged in any other suitable method for final distribution.

The base unpurified saline solution is prepared by boiling distilled purified water and adding dry powder sea salt to the water to achieve the desired concentration of 2.5 grams salt per deciliter. The solution is then tested for concentration and pH.

The lactoferrin solution is prepared by adding the powdered lactoferrin to the warm saline solution, that when in liquid form is added to the carrier saline solution to achieve the desired concentration in the range of 0.002% to 1.5% by volume. Calcium chloride powder is added to the warm saline solution until dissolved and then added to the carrier saline solution to a concentration in the range of 0.61% to 4.7% by volume. Sodium bicarbonate powder is added at the end to the final solution to achieve a pH in the range of 7.3 to 7.45.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A wound cleaning chemical composition, consisting of:
an unpurified saline solution;
a lactoferrin solution;
an ethanol solution;
a sodium bicarbonate solution; and
a hypochlorite solution.

2. The wound cleaning chemical composition according to claim 1, wherein the unpurified saline solution is prepared by boiling distilled purified water and adding dry powder sea salt to the boiling distilled purified water.

3. The wound cleaning chemical composition according to claim 2, wherein the unpurified saline solution is tested for concentration and pH.

4. The wound cleaning chemical composition according to claim 3, wherein the pH is in the range of 7.3 to 7.45.

5. The wound cleaning chemical composition according to claim 1, wherein the wound cleaning chemical composition is heated.

6. The wound cleaning chemical composition according to claim 1, wherein the wound cleaning chemical composition is tested for osmolality.

7. The wound cleaning chemical composition according to claim 1, wherein the wound cleaning chemical composition is tested for bacterial count.

8. A wound cleaning chemical composition, consisting of:
an unpurified saline solution in the range of 1.5 to 3 grams per cc;
a lactoferrin solution in the range of 0.001% to 1.5% by volume;
an ethanol solution in the range of 0.05% to 1.5% by volume;
a sodium bicarbonate solution in the range of 1.2% to 5.1% by volume; and
a hypochlorite solution in the range of 0.06% to 4.7% by volume.

9. The wound cleaning chemical composition according to claim 8, wherein the unpurified saline solution is prepared by boiling distilled purified water and adding dry powder sea salt to the boiling distilled purified water.

10. The wound cleaning chemical composition according to claim 9, wherein the unpurified saline solution is tested for concentration and pH.

11. The wound cleaning chemical composition according to claim 10, wherein the pH is in the range of 7.3 to 7.45.

12. The wound cleaning chemical composition according to claim 8, wherein the wound cleaning chemical composition is heated.

13. The wound cleaning chemical composition according to claim 12, wherein the wound cleaning chemical composition is pasteurized.

14. The wound cleaning chemical composition according to claim 8, wherein the wound cleaning chemical composition is tested for osmolality.

15. The wound cleaning chemical composition according to claim 8, wherein the wound cleaning chemical composition is tested for bacterial count.

16. A method for manufacturing a wound cleaning chemical composition, consisting of the steps:
preparing an unpurified saline solution;
adding a hypochlorite solution, an ethanol solution and an sodium bicarbonate solution to the unpurified saline solution; and
adding a lactoferrin solution.

17. The method for manufacturing a wound cleaning chemical composition according to claim 16, wherein the unpurified saline solution is prepared by boiling distilled purified water and adding dry powder sea salt to the boiling distilled purified water.

18. The method for manufacturing a wound cleaning chemical composition according to claim 16, wherein the wound cleaning chemical composition is pasteurized.

19. The method for manufacturing a wound cleaning chemical composition according to claim 18, wherein the wound cleaning chemical composition is tested to achieve a pH in the range of 7.3 to 7.45.

20. The method for manufacturing a wound cleaning chemical composition according to claim 19, wherein the wound cleaning chemical composition is cooled.
